(19) United States
(12) Patent Application Publication
(10) Pub. No.: US 2026/0263031 A1
(43) Pub. Date: Sep. 10, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Kaito TAKASHIMA, Kanagawa (JP)

(73) Assignee: FUJIFILM Corporation, Tokyo (JP)

(21) Appl. No.: 19/553,417

(22) Filed: Mar. 1, 2026

(30) Foreign Application Priority Data
Mar. 6, 2025 (JP) 2025-035582

(51) Int. Cl. A61B 8/08; A61B 5/00; A61B 8/00; A61B 8/06

(52) U.S. Cl. CPC A61B 8/0883; A61B 5/742; A61B 8/065; A61B 8/4483; A61B 8/461

(57) ABSTRACT

Provided are an ultrasound diagnostic apparatus and a control method of an ultrasound diagnostic apparatus that can accurately display first and second ultrasound images that are consistent with each other.

The ultrasound diagnostic apparatus includes an image acquisition unit that acquires a first ultrasound image and a second ultrasound image in which first and second cross sections of a heart of a subject that are different from each other are imaged, respectively, a first measurement unit that acquires a first measurement value of a predetermined measurement target depicted in the first cross section from the first ultrasound image, a second measurement unit that acquires a second measurement value of the predetermined measurement target depicted in the second cross section from the second ultrasound image, a consistency calculation unit that calculates consistency between the first ultrasound image and the second ultrasound image by comparing the first measurement value and the second measurement value, and a notification unit that notifies a user of the consistency.

### CROSS-REFERENCE TO RELATED APPLICATIONS

The present application claims priority under 35 U.S.C. § 119 to Japanese Patent Application No. 2025-035582, filed on Mar. 6, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method of the ultrasound diagnostic apparatus.

### 2. Description of the Related Art

In the related art, a so-called cardiac output is calculated by capturing an ultrasound image representing a tomographic plane of a heart of a subject using a so-called ultrasound diagnostic apparatus and analyzing the captured ultrasound image. The cardiac output is usually calculated by performing calculation steps of (1) measuring a diameter of a left ventricular outflow tract in a first ultrasound image representing a so-called parasternal left ventricular long-axis cross section of a systolic mid-phase of the heart, and calculating a cross-sectional area of the left ventricular outflow tract, (2) calculating a velocity-time integral value of blood flow in the left ventricular outflow tract using a second ultrasound image representing a so-called apical five-chamber cross section or a so-called apical three-chamber cross section by a so-called pulse Doppler method, (3) calculating a so-called stroke volume as a product of the cross-sectional area of the left ventricular outflow tract and the velocity-time integral value of the blood flow in the left ventricular outflow tract, and (4) calculating the cardiac output as a product of the stroke volume and a heart rate.

As described above, since the measurement of the stroke volume and the cardiac output requires a plurality of procedures, various techniques have been developed to easily measure the stroke volume and the cardiac output. For example, JP6987048B discloses that a region of interest is automatically set in a left ventricular outflow tract in an ultrasound image, a plurality of candidates of a Doppler gate are set in the set region of interest, and an optimal position of the Doppler gate is selected based on a Doppler spectrum waveform calculated for each of the plurality of candidates of the Doppler gate.

### SUMMARY OF THE INVENTION

However, for example, in a case in which the second ultrasound image representing the apical five-chamber cross section or the apical three-chamber cross section is not appropriately depicted, even in a case in which the technique of JP6987048B is used, the Doppler gate cannot be set at an appropriate position, and as a result, an appropriate time-varying waveform of the blood flow velocity measured in the Doppler gate may not be obtained. In this case, the user often repeats a series of operations of adjusting a position of an ultrasound probe to a position at which the second ultrasound image can be appropriately depicted, setting the Doppler gate in the second ultrasound image, and checking the time-varying waveform of the blood flow velocity obtained in this manner, and it may take a large amount of time to obtain an appropriate second ultrasound image.

The present invention has been made to solve such problems in the related art, and an object of the present invention is to provide an ultrasound diagnostic apparatus and a control method of an ultrasound diagnostic apparatus that can accurately depict first and second ultrasound images that are consistent with each other.

The above object can be achieved with the following configurations.

[1] An ultrasound diagnostic apparatus including: an image acquisition unit that acquires a first ultrasound image and a second ultrasound image in which first and second cross sections of a heart of a subject that are different from each other are imaged, respectively; a first measurement unit that acquires a first measurement value of a predetermined measurement target depicted in the first cross section from the first ultrasound image; a second measurement unit that acquires a second measurement value of the predetermined measurement target depicted in the second cross section from the second ultrasound image; a consistency calculation unit that calculates consistency between the first ultrasound image and the second ultrasound image by comparing the first measurement value acquired by the first measurement unit and the second measurement value acquired by the second measurement unit; and a notification unit that notifies a user of the consistency calculated by the consistency calculation unit.

[2] The ultrasound diagnostic apparatus according to [1], in which the image acquisition unit acquires the first ultrasound image in which a parasternal left ventricular long-axis cross section is set as the first cross section and the second ultrasound image in which an apical five-chamber cross section or an apical three-chamber cross section is set as the second cross section, and the first measurement value and the second measurement value are obtained by measuring a left ventricular outflow tract diameter as the first measurement value and the second measurement value, respectively.

[3] The ultrasound diagnostic apparatus according to [1] or [2], in which the consistency calculation unit calculates a difference or a ratio between the first measurement value and the second measurement value as the consistency.

[4] The ultrasound diagnostic apparatus according to any one of [1] to [3], further including: a monitor, in which the notification unit displays the consistency calculated by the consistency calculation unit on the monitor.

[5] The ultrasound diagnostic apparatus according to any one of [1] to [3], further including: a speaker, in which the notification unit outputs the consistency calculated by the consistency calculation unit as sound from the speaker.

[6] The ultrasound diagnostic apparatus according to any one of [1] to [3], further including: a warning lamp, in which the notification unit causes the warning lamp to emit light in a case in which the consistency calculated by the consistency calculation unit is lower than a predetermined threshold value.

[7] The ultrasound diagnostic apparatus according to any one of [1] to [3], further including: an ultrasound probe, in which the ultrasound probe has a built-in vibrator, and the notification unit causes the vibrator to vibrate in a case in which the consistency calculated by the consistency calculation unit is lower than a predetermined threshold value.

[8] The ultrasound diagnostic apparatus according to any one of [1] to [7], further including: a velocity-time integral value calculation unit that calculates a velocity-time integral value of blood flow in a left ventricular outflow tract from the second ultrasound image; and a cardiac output calculation unit that calculates a cardiac output based on the left ventricular outflow tract diameter as the first measurement value acquired by the first measurement unit and the velocity-time integral value calculated by the velocity-time integral value calculation unit, in which the notification unit notifies the user in a case in which the cardiac output calculated by the cardiac output calculation unit deviates from a predetermined reference value.

[9] A control method of an ultrasound diagnostic apparatus, the method including: acquiring a first ultrasound image and a second ultrasound image in which first and second cross sections of a heart of a subject that are different from each other are imaged, respectively; acquiring a first measurement value of a predetermined measurement target depicted in the first cross section from the first ultrasound image; acquiring a second measurement value of the predetermined measurement target depicted in the second cross section from the second ultrasound image; calculating consistency between the first ultrasound image and the second ultrasound image by comparing the first measurement value and the second measurement value that are acquired; and notifying a user of the calculated consistency.

According to the present invention, the ultrasound diagnostic apparatus includes the image acquisition unit that acquires the first ultrasound image and the second ultrasound image in which first and second cross sections of the heart of the subject that are different from each other are imaged, respectively, the first measurement unit that acquires the first measurement value of the predetermined measurement target depicted in the first cross section from the first ultrasound image, the second measurement unit that acquires the second measurement value of the predetermined measurement target depicted in the second cross section from the second ultrasound image, the consistency calculation unit that calculates consistency between the first ultrasound image and the second ultrasound image by comparing the first measurement value acquired by the first measurement unit and the second measurement value acquired by the second measurement unit, and the notification unit that notifies the user of the consistency calculated by the consistency calculation unit. With this configuration, the first ultrasound image and the second ultrasound image that are consistent with each other can be accurately displayed.

### BRIEF DESCRIPTION OF THE DRAWINGS

FIG. 1 is a block diagram showing a configuration of an ultrasound diagnostic apparatus according to an embodiment of the present invention.

FIG. 2 is a block diagram showing an internal configuration of a transmission and reception circuit in an embodiment of the present invention.

FIG. 3 is a block diagram showing an internal configuration of an image generation unit in an embodiment of the present invention.

FIG. 4 is a diagram showing an example of a first ultrasound image representing a parasternal left ventricular long-axis cross section.

FIG. 5 is a diagram showing an example of a second ultrasound image representing an apical five-chamber cross section.

FIG. 6 is a diagram showing an example of a second ultrasound image representing an apical three-chamber cross section.

FIG. 7 is a diagram showing a display example of a consistency between the first ultrasound image and the second ultrasound image on a monitor.

FIG. 8 is a block diagram showing an internal configuration of a velocity-time integral value calculation unit.

FIG. 9 is a flowchart showing an operation of the ultrasound diagnostic apparatus according to the embodiment of the present invention.

FIG. 10 is a block diagram showing a partial configuration of a first modification example of the ultrasound diagnostic apparatus according to the embodiment of the present invention.

FIG. 11 is a block diagram showing a partial configuration of a second modification example of the ultrasound diagnostic apparatus according to the embodiment of the present invention.

FIG. 12 is a block diagram showing a partial configuration of a third modification example of the ultrasound diagnostic apparatus according to the embodiment of the present invention.

### DESCRIPTION OF THE PREFERRED EMBODIMENTS

Hereinafter, embodiments of the present invention will be described based on the accompanying drawings.

The following configuration requirements are described based on a representative embodiment of the present invention, but the present invention is not limited to the embodiment.

In the present specification, a numerical range represented by “to” means a range including numerical values described before and after “to”, both ends inclusive, as a lower limit value and an upper limit value.

In the present specification, “the same” includes an error range generally allowed in the technical field.

### EMBODIMENT

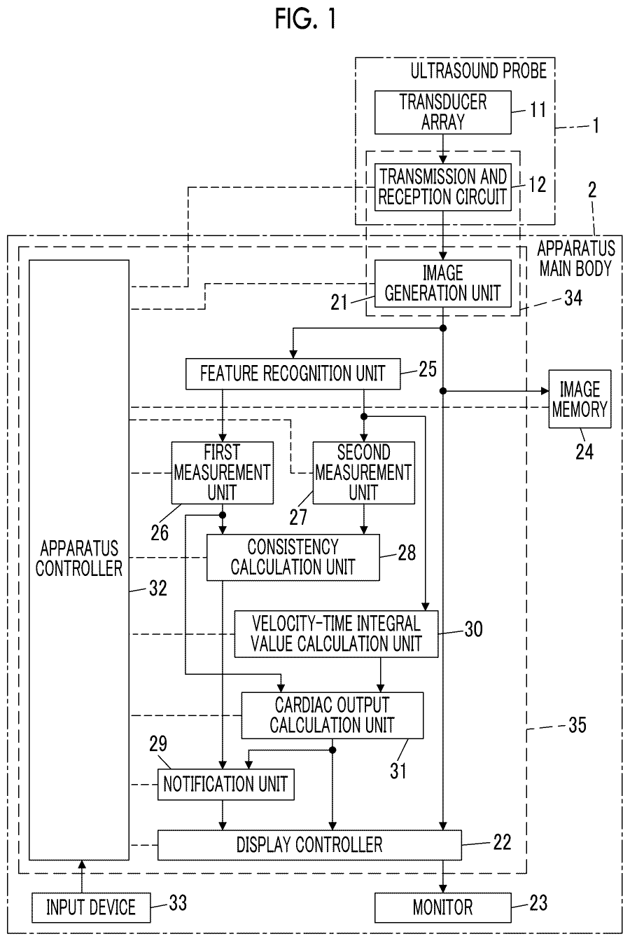

FIG. 1 shows a configuration of an ultrasound diagnostic apparatus according to an embodiment of the present invention. The ultrasound diagnostic apparatus comprises an ultrasound probe 1 and an apparatus main body 2 that are connected to each other by so-called wired communication or so-called wireless communication.

The ultrasound probe 1 comprises a transducer array 11 and a transmission and reception circuit 12 connected to the transducer array 11.

The apparatus main body 2 comprises an image generation unit 21 connected to the transmission and reception circuit 12. In the apparatus main body 2, a display controller 22 and a monitor 23 are sequentially connected to the image generation unit 21. In addition, an image memory 24 and an image recognition unit 25 are connected to the image generation unit 21. A first measurement unit 26 and a second measurement unit 27 are connected to the image recognition unit 25. A consistency calculation unit 28 is connected to the first measurement unit 26 and the second measurement unit 27. A notification unit 29 is connected to the consistency calculation unit 28. In addition, a velocity-time integral value calculation unit 30 is connected to the image recognition unit 25. A cardiac output calculation unit 31 is connected to the velocity-time integral value calculation unit 30. The cardiac output calculation unit 31 is connected to the display controller 22 and the notification unit 29. In addition, an apparatus controller 32 is connected to the transmission and reception circuit 12, the image generation unit 21, the display controller 22, the image recognition unit 25, the first measurement unit 26, the second measurement unit 27, the consistency calculation unit 28, the notification unit 29, the velocity-time integral value calculation unit 30, and the cardiac output calculation unit 31. An input device 33 is connected to the apparatus controller 32.

The transmission and reception circuit 12 and the image generation unit 21 constitute an image acquisition unit 34. In addition, the image generation unit 21, the display controller 22, the image recognition unit 25, the first measurement unit 26, the second measurement unit 27, the consistency calculation unit 28, the notification unit 29, the velocity-time integral value calculation unit 30, the cardiac output calculation unit 31, and the apparatus controller 32 constitute a processor 35 for the apparatus main body 2.

The transducer array 11 of the ultrasound probe 1 has a plurality of ultrasound transducers arranged in a one-dimensional or two-dimensional manner. In accordance with a drive signal supplied from the transmission and reception circuit 12, each of the ultrasound transducers transmits ultrasound and receives an ultrasound echo from a subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 34, which is composed of the transmission and reception circuit 12 and the image generation unit 21, acquires ultrasound images of a plurality of frames as a moving image in which a heart of the subject is imaged, by transmitting and receiving ultrasound beams using the ultrasound probe 1.

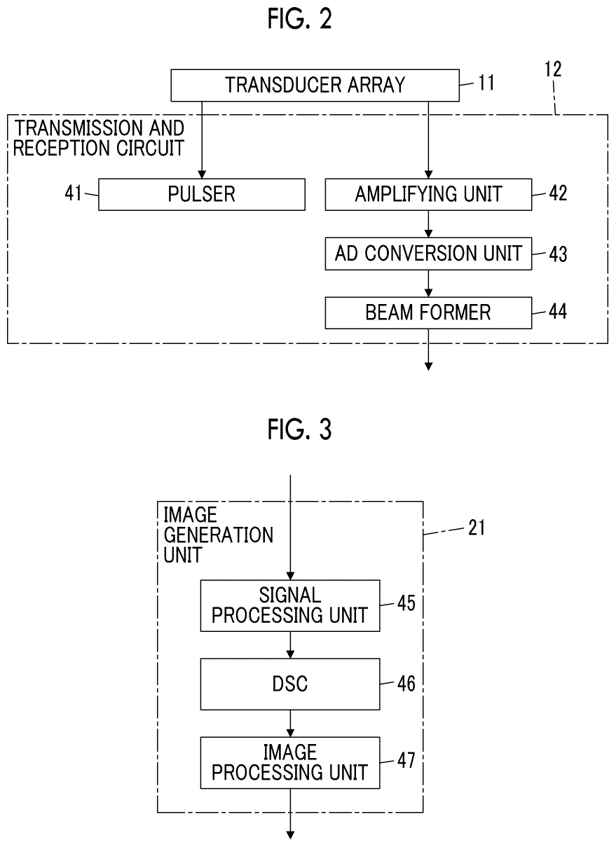

The transmission and reception circuit 12 transmits the ultrasound waves from the transducer array 11 and generates a sound ray signal based on reception signals acquired by the transducer array 11 under control of the apparatus controller 32. As shown in FIG. 2, the transmission and reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus controller 32 so that the ultrasound waves transmitted from the plurality of ultrasound transducers of the transducer array 11 form an ultrasound beam, and supplies each drive signal to the plurality of ultrasound transducers. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplifying unit 42.

The amplifying unit 42 amplifies the signal input from each of the ultrasound transducers constituting the transducer array 11 and transmits the amplified signal to the AD conversion unit 43. The AD conversion unit 43 converts the signal transmitted from the amplifying unit 42 into digital reception data. The beam former 44 performs so-called reception focus processing by applying and adding the delay to each reception data received from the AD conversion unit 43. By the reception focus processing, each reception data, which is converted by the AD conversion unit 43, is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation by distance of the sound ray signal received from the transmission and reception circuit 12 in accordance with depths of reflection positions of the ultrasound waves using a sound speed value set by the apparatus controller 32 and then performs envelope detection processing on the sound ray signal to generate a B-mode image signal that is tomographic image information related to tissues inside the subject.

The DSC 46 converts (raster-converts) the B-mode image signal, which is generated by the signal processing unit 45, into the image signal in accordance with a normal television signal scanning method.

The image processing unit 47 performs various types of necessary image processing such as gradation processing on the B-mode image signal input from the DSC 46, and then transmits the B-mode image signal to the display controller 22, the image memory 24, and the image recognition unit 25. Hereinafter, the B-mode image signal, which is image-processed by the image processing unit 47, will be referred to as an ultrasound image.

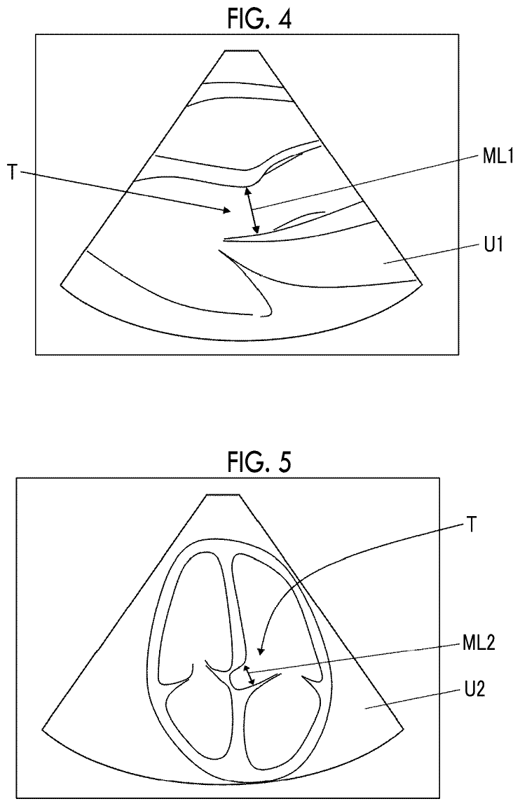

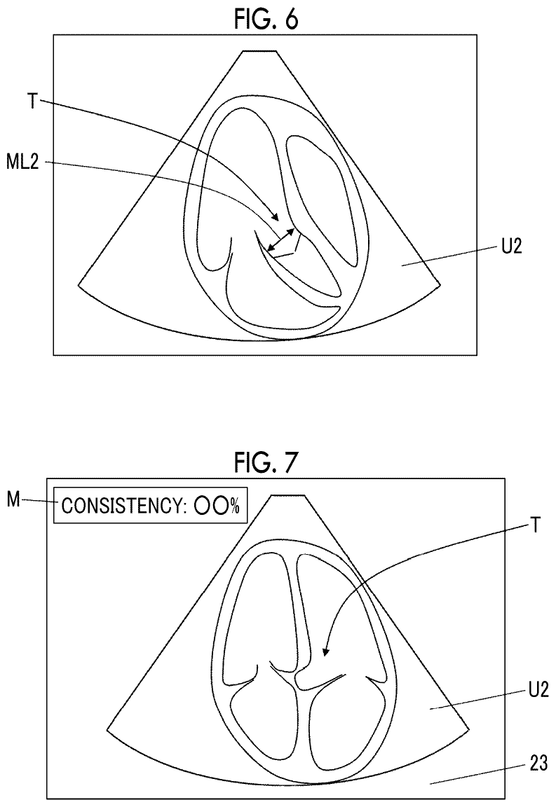

In the present invention, the image acquisition unit 34 acquires a first ultrasound image and a second ultrasound image in which first and second cross sections of the heart of the subject that are different from each other are imaged, respectively. For example, as schematically shown in FIG. 4, the image acquisition unit 34 can acquire an ultrasound image representing a so-called parasternal left ventricular long-axis cross section as the first ultrasound image U1 representing the first cross section. The parasternal left ventricular long-axis cross section is a cross section including a left ventricle, a left ventricular outflow tract T, a left atrium, and a right ventricle. In addition, for example, as schematically shown in FIGS. 5 and 6, the image acquisition unit 34 can acquire an ultrasound image representing a so-called apical five-chamber cross section or an ultrasound image representing a so-called apical three-chamber cross section as the second ultrasound image U2 representing the second cross section. The apical five-chamber cross section is a cross section of the heart including five lumens of the left ventricle, the left ventricular outflow tract, the left atrium, the right ventricle, and the right atrium. In addition, the apical three-chamber cross section is a cross section of the heart including three lumens of the left ventricle, the left atrium, and the right ventricle. Both the apical five-chamber cross section and the apical three-chamber cross section include the left ventricular outflow tract T.

Hereinafter, a main example will be described in which the image acquisition unit 34 acquires the ultrasound image representing the parasternal left ventricular long-axis cross section as the first ultrasound image U1 and acquires the ultrasound image representing the apical five-chamber cross section or the ultrasound image representing the apical three-chamber cross section as the second ultrasound image U2, but the image acquisition unit 34 can also acquire the ultrasound image representing the apical five-chamber cross section or the ultrasound image representing the apical three-chamber cross section as the first ultrasound image U1 and acquire the ultrasound image representing the parasternal left ventricular long-axis cross section as the second ultrasound image U2.

The image memory 24 is a memory that stores the first ultrasound image U1 and the second ultrasound image U2 that are acquired by the image acquisition unit 34. The user can use the first ultrasound image U1 and the second ultrasound image U2 stored in the image memory 24, for example, to check the first ultrasound image U1 and the second ultrasound image U2 after the examination. For example, a recording medium, such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory), can be used as the image memory 24.

The image recognition unit 25 recognizes any of the first cross section or the second cross section, that is, any of the parasternal left ventricular long-axis cross section, the apical five-chamber cross section, or the apical three-chamber cross section from the ultrasound image acquired by the image acquisition unit 34. The image recognition unit 25 can recognize any of the parasternal left ventricular long-axis cross section, the apical five-chamber cross section, or the apical three-chamber cross section by using, for example, a so-called template matching method of searching the ultrasound image using template data representing a typical image pattern of the parasternal left ventricular long-axis cross section, the apical five-chamber cross section, and the apical three-chamber cross section, in which the template data is stored in advance. The image recognition unit 25 has, for example, a trained model in so-called machine learning, which is trained learning is performed using a large number of ultrasound images representing the parasternal left ventricular long-axis cross section, the apical five-chamber cross section, and the apical three-chamber cross section, and can also cause the trained model to output a recognition result by inputting the ultrasound image to the trained model.

The image recognition unit 25 transmits, for example, the first ultrasound image U1 in which the parasternal left ventricular long-axis cross section is recognized to the first measurement unit 26, and transmits the second ultrasound image U2 representing the apical five-chamber cross section or the apical three-chamber cross section to the second measurement unit 27 and the velocity-time integral value calculation unit 30.

The first measurement unit 26 acquires the first measurement value of the predetermined measurement target depicted in the first cross section from the first ultrasound image U1. The first measurement unit 26 can measure, for example, a diameter of the left ventricular outflow tract T depicted in the parasternal left ventricular long-axis cross section, that is, a left ventricular outflow tract diameter as the first measurement value of the predetermined measurement target depicted in the first cross section. In this case, the first measurement unit 26 can measure, for example, as shown in FIG. 4, a length of a measurement line ML1 disposed on the left ventricular outflow tract T as the left ventricular outflow tract diameter. The first measurement unit 26 can dispose the measurement line ML1 at a position designated by the user on the first ultrasound image U1 via the input device 33, for example.

The second measurement unit 27 acquires the second measurement value of the predetermined measurement target depicted in the second cross section from the second ultrasound image U2. The predetermined measurement target in the second cross section is the same as the predetermined measurement target in the first cross section. The second measurement unit 27 can measure, for example, a diameter of the left ventricular outflow tract T depicted in the apical five-chamber cross section or the apical three-chamber cross section, that is, a left ventricular outflow tract diameter as the second measurement value of the predetermined measurement target depicted in the second cross section. In this case, the second measurement unit 27 can measure, for example, as shown in FIGS. 5 and 6, a length of a measurement line ML2 disposed on the left ventricular outflow tract T as the left ventricular outflow tract diameter. The second measurement unit 27 can dispose the measurement line ML2 at a position designated by the user on the second ultrasound image U2 via the input device 33, for example.

Here, the left ventricular outflow tract diameter on the parasternal left ventricular long-axis cross section and the left ventricular outflow tract diameter on the apical five-chamber cross section are ideally equal to each other, and the left ventricular outflow tract diameter on the parasternal left ventricular long-axis cross section and the left ventricular outflow tract diameter on the apical three-chamber cross section are also ideally equal to each other. In this way, as the measurement target on the first cross section and the second cross section, a target object is set such that the first measurement value and the second measurement value are ideally equal to each other.

The consistency calculation unit 28 calculates the consistency between the first ultrasound image U1 and the second ultrasound image U2 by comparing the first measurement value acquired by the first measurement unit 26 and the second measurement value acquired by the second measurement unit 27. The consistency calculation unit 28 can calculate a difference or a ratio between the first measurement value and the second measurement value as the consistency between the first ultrasound image U1 and the second ultrasound image U2. In addition, the consistency calculation unit 28 can calculate the consistency by Expression (1) shown below by setting the first measurement value as MV1, the second measurement value as MV2, and the value of the consistency as J [%].

$$J = 100 - min \{ 100 , 100 \times \vert \frac{MV 1 - MV 2}{( \frac{MV 1 + MV 2}{2} )} \vert \} \qquad ( 1 )$$

The function min (A, B) in Expression (1) is a function that outputs a smaller value between the value A and the value B. For example, in a case in which the value A>the value B, min (A, B)=B.

Since the first measurement value and the second measurement value are ideally equal to each other, the closer the difference between the first measurement value and the second measurement value is to 0, and the closer the ratio between the first measurement value and the second measurement value is to 1, the higher the consistency between the first ultrasound image U1 and the second ultrasound image U2 is considered to be. On the contrary, the larger the absolute value of the difference between the first measurement value and the second measurement value is, and the larger the ratio between the first measurement value and the second measurement value is than 1 or the closer the ratio is to 0, the lower the consistency between the first ultrasound image U1 and the second ultrasound image U2 is considered to be. In addition, the value J of the consistency calculated by Expression (1) is a value close to 100 as the first measurement value and the second measurement value are close to each other, and is a value close to 0 as the difference between the first measurement value and the second measurement value is large.

The high value of the consistency calculated by the consistency calculation unit 28 is considered to be that the second ultrasound image U2 having high consistency with respect to the first ultrasound image U1 is acquired, that is, the second ultrasound image U2 is appropriately acquired. In addition, the low value of the consistency is considered to be that the second ultrasound image U2 is not appropriately acquired.

The notification unit 29 notifies the user of the consistency calculated by the consistency calculation unit 28. The notification unit 29 can display the consistency on the monitor 23 in a form of a message M for notifying the consistency, for example, as shown in FIG. 7. The notification unit 29 can store a consistency threshold value set for the consistency, and, in a case in which the consistency calculated by the consistency calculation unit 28 is lower than the consistency threshold value, can display, on the monitor 23, a message M or the like indicating that the second ultrasound image U2 is to be reacquired by moving, inclining, or rotating the ultrasound probe 1. The notification unit 29 can display, on the monitor 23, a message M or the like indicating that the second ultrasound image U2 is appropriately acquired, in a case in which the consistency is equal to or higher than the consistency threshold value.

In general, in order to check whether the second ultrasound image U2 representing the apical five-chamber cross section or the apical three-chamber cross section is appropriately acquired, a series of operations of installing a so-called Doppler gate on the left ventricular outflow tract T in the second ultrasound image U2, acquiring a time-varying waveform of the blood flow velocity in the left ventricular outflow tract T using a so-called pulse Doppler method in the Doppler gate, and checking the time-varying waveform is often repeated, and it may take a large amount of time to obtain an appropriate second ultrasound image U2. The user can easily ascertain whether or not the second ultrasound image U2 is appropriately acquired by checking the consistency notified by the notification unit 29, and can accurately display the second ultrasound image U2.

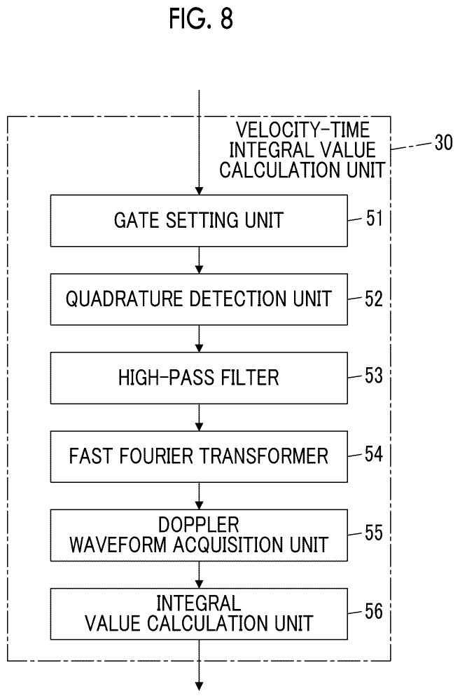

The velocity-time integral value calculation unit 30 calculates the velocity-time integral value of the blood flow in the left ventricular outflow tract T, that is, the blood flow in the left ventricular outflow tract from the second ultrasound image U2. As shown in FIG. 8, the velocity-time integral value calculation unit 30 has a configuration in which a gate setting unit 51, a quadrature detection unit 52, a high-pass filter 53, a fast Fourier transformer 54, a Doppler waveform acquisition unit 55, and an integral value calculation unit 56 are connected in series.

The gate setting unit 51 sets a so-called Doppler gate used to calculate the velocity-time integral value of the blood flow by a so-called pulse Doppler method on the ultrasound image U acquired by the image acquisition unit 34. The gate setting unit 51 sets the Doppler gate on the left ventricular outflow tract T on the second ultrasound image U2, for example.

The gate setting unit 51 can set the Doppler gate at a position designated by the user via the input device 33, for example. In addition, the gate setting unit 51 can recognize the left ventricular outflow tract T shown in the second ultrasound image U2 by analyzing the second ultrasound image U2, and set the Doppler gate on the recognized left ventricular outflow tract T, for example. In this case, the gate setting unit 51 can recognize the left ventricular outflow tract T by, for example, a so-called template matching method of searching the second ultrasound image U2 using a typical image pattern of the left ventricular outflow tract T on the apical five-chamber cross section and the apical three-chamber cross section, which is stored, or a method of using a trained model in machine learning trained on the image pattern of the left ventricular outflow tract T on the apical five-chamber cross section and the apical three-chamber cross section.

The quadrature detection unit 52 performs quadrature detection on the sound ray signal by mixing the sound ray signal received from the transmission and reception circuit 12 with a carrier signal of a reference frequency, and converts the sound ray signal into a complex signal.

The high-pass filter 53 functions as a so-called wall filter, and removes a frequency component derived from the motion of the body tissue inside the subject, from the complex signal generated by the quadrature detection unit 52.

The fast Fourier transformer 54 performs a Fourier transform on the complex data of a plurality of sample points to perform frequency analysis, obtains the blood flow velocity, and generates a spectrum signal.

The Doppler waveform acquisition unit 55 acquires a Doppler waveform image signal by representing the magnitude of each frequency component with brightness while aligning the spectrum signal generated by the fast Fourier transform unit 54 on a time axis. The Doppler waveform image signal is called a so-called Doppler image, represents the time-varying waveform of the blood flow velocity, shows a time axis on the horizontal axis, shows a Doppler shift frequency, that is, the flow velocity on the vertical axis, and represents the power in each frequency component with the brightness of the waveform.

The integral value calculation unit 56 specifies a cardiac cycle period in the Doppler waveform image signal, and calculates the velocity-time integral value by time-integrating the value of the flow velocity in the Doppler waveform image signal for the cardiac cycle period. The integral value calculation unit 56 can specify a repeating unit of the waveform in the Doppler waveform image signal by analyzing the Doppler waveform image signal, and can specify a period to which the specified repeating unit belongs as the cardiac cycle period, for example.

Since the Doppler gate is set on the left ventricular outflow tract T on the second ultrasound image U2 by the gate setting unit 51, the velocity-time integral value calculated by the velocity-time integral value calculation unit 30 in this way is a value obtained by integrating the velocity of the blood flow flowing through the left ventricular outflow tract T for the cardiac cycle period. The velocity-time integral value can be used to calculate a so-called stroke volume and a cardiac output together with the left ventricular outflow tract diameter measured in the ultrasound image representing the so-called parasternal left ventricular long-axis cross section or the like.

The processing of calculating the velocity-time integral value by the quadrature detection unit 52, the high-pass filter 53, the fast Fourier transformer 54, the Doppler waveform acquisition unit 55, and the integral value calculation unit 56 is performed after the transducer array 11 is driven by transmitting the drive signal under the control of the transmission and reception circuit 12 and the apparatus controller 32 to transmit the ultrasound wave in a pulse shape.

The cardiac output calculation unit 31 calculates the cardiac output based on the left ventricular outflow tract diameter as the first measurement value acquired by the first measurement unit 26 and the velocity-time integral value calculated by the velocity-time integral value calculation unit 30. More specifically, the cardiac output calculation unit 31 calculates a so-called stroke volume by a product of the left ventricular outflow tract diameter and the velocity-time integral value, and calculates the cardiac output by a product of the calculated stroke volume and the heart rate. The cardiac output calculation unit 31 can calculate, for example, the reciprocal of the period of the specified repeating waveform as the heart rate by specifying the repeating waveform in the time-varying waveform of the blood flow velocity acquired by the Doppler waveform acquisition unit 55 of the velocity-time integral value calculation unit 30.

In a case in which the cardiac output calculated by the cardiac output calculation unit 31 deviates from a reference value, the notification unit 29 can notify the user that the cardiac output indicates an abnormal value, the second ultrasound image U2 is not appropriately depicted, and the like. The reference value of the cardiac output can be set, for example, in a range of 2500 mL to 4500 mL. The deviation of the cardiac output from the reference value may occur, for example, in a case in which blood regurgitates in the aorta of the heart due to a heart disease.

The display controller 22 performs predetermined processing on the first ultrasound image U1 and the second ultrasound image U2 acquired by the image acquisition unit 34, the content of the notification by the notification unit 29, and the stroke volume or the cardiac output calculated by the cardiac output calculation unit 31, under the control of the apparatus controller 32, and displays the results on the monitor 23.

The monitor 23 displays the first ultrasound image U1 and the second ultrasound image U2, the content of the notification by the notification unit 29, and the stroke volume or the cardiac output calculated by the cardiac output calculation unit 31 under the control of the display controller 22, and has, for example, a display device such as a liquid crystal display (LCD) or an organic electroluminescence display (organic EL display).

The input device 33 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing in the processor 35 is executed by any computer. In addition, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the order described above and may be changed as appropriate. Any computer may be a general-purpose computer, a computer for a specific use, a workstation, or another system capable of executing each process.

The processor 35 may be configured by one or a plurality of hardware, and the type of hardware is not limited. For example, the processor 35 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). In addition, the types of hardware may be a combination of different types of hardware. In a case where a plurality of hardware are configured to execute one or a plurality of processes of a certain processor, the plurality of hardware may be present in devices physically separated from each other, or may be present in the same device. In addition, in any of the embodiments, the order of each processing by the processor 35 is not limited to the above order, and may be changed as appropriate. The hardware is composed of an electric circuit (circuitry) in which circuit elements such as semiconductor elements are combined.

Furthermore, the program may be software such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be stored in a plurality of non-transitory computer-readable media existing in devices physically separated from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

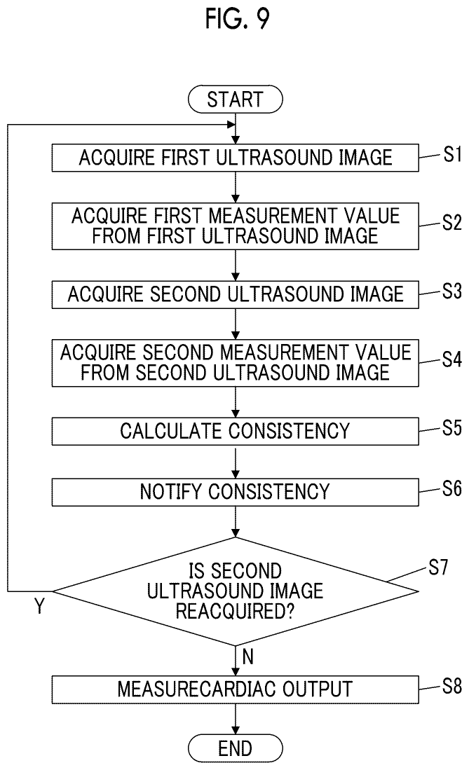

Hereinafter, an operation of the ultrasound diagnostic apparatus according to the embodiment will be described with reference to a flowchart shown in FIG. 9. Here, an example will be described in which the ultrasound image representing the parasternal left ventricular long-axis cross section is acquired as the first ultrasound image U1, and the ultrasound image representing the apical five-chamber cross section is acquired as the second ultrasound image U2.

In step S1, the image acquisition unit 34 acquires the first ultrasound image U1 representing the parasternal left ventricular long-axis cross section. In such a case, under the control of the apparatus controller 32, the transmission and reception of the ultrasound from the plurality of transducers of the transducer array 11 are started in accordance with the drive signal from the pulser 41 of the transmission and reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus main body 2, and thus the ultrasound image is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing, such as gradation processing. The first ultrasound image U1 generated in step S1 in this way is transmitted to the display controller 22 and the first measurement unit 26.

In step S2, the first measurement unit 26 acquires the left ventricular outflow tract diameter as the first measurement value of the predetermined measurement target from the first ultrasound image U1 acquired in step S1. The first measurement unit 26 can measure, for example, as shown in FIG. 4, a length of a measurement line ML1 disposed on the left ventricular outflow tract T as the left ventricular outflow tract diameter. The first measurement unit 26 can dispose the measurement line ML1 at a position designated by the user on the first ultrasound image U1 via the input device 33, for example.

In step S3, the user adjusts the ultrasound probe 1 to a position and a posture for capturing the second ultrasound image U2 representing the apical five-chamber cross section by moving and inclining the ultrasound probe 1, and then the image acquisition unit 34 acquires the second ultrasound image U2 representing the apical five-chamber cross section. Since the processing of generating the second ultrasound image U2 is the same as the processing of generating the first ultrasound image U1 in step S1, the description of the processing of generating the second ultrasound image U2 will be omitted.

In step S4, the second measurement unit 27 acquires the left ventricular outflow tract diameter as the second measurement value of the predetermined measurement target from the second ultrasound image U2 acquired in step S3. The second measurement unit 27 can measure, for example, as shown in FIG. 5, a length of a measurement line ML2 disposed on the left ventricular outflow tract T as the left ventricular outflow tract diameter. The second measurement unit 27 can dispose the measurement line ML2 at a position designated by the user on the second ultrasound image U2 via the input device 33, for example.

In step S5, the consistency calculation unit 28 calculates the consistency between the first ultrasound image U1 and the second ultrasound image U2 by comparing the first measurement value acquired in step S2 and the second measurement value acquired in step S4. The consistency calculation unit 28 can calculate a difference or a ratio between the first measurement value and the second measurement value as the consistency between the first ultrasound image U1 and the second ultrasound image U2. In addition, the consistency calculation unit 28 can also calculate the consistency by using Expression (1).

In step S6, the notification unit 29 notifies the user of the consistency calculated in step S5. For example, as shown in FIG. 7, the notification unit 29 can notify the user of the consistency by displaying the message M on the monitor 23. In addition, the notification unit 29 can also notify the user of whether or not the second ultrasound image U2 is appropriately acquired by comparing the value of the consistency calculated in step S5 and the consistency threshold value stored in advance. The user can check the notification content of the notification unit 29 to determine whether or not the second ultrasound image U2 is appropriately acquired, or whether or not it is necessary to adjust the position and the posture of the ultrasound probe 1 in order to appropriately acquire the second ultrasound image U2.

In step S7, the apparatus controller 32 determines whether or not to reacquire the second ultrasound image U2. For example, in a case in which the user checks the notification content in step S6, determines that the second ultrasound image U2 is not appropriately acquired, and inputs an instruction to reacquire the second ultrasound image U2 via the input device 33, the apparatus controller 32 determines to reacquire the second ultrasound image U2. For example, in a case in which the user checks the notification content in step S6, determines that the second ultrasound image U2 is appropriately acquired, and inputs an instruction not to reacquire the second ultrasound image U2 via the input device 33, the apparatus controller 32 determines not to reacquire the second ultrasound image U2.

In a case in which it is determined to reacquire the second ultrasound image U2 in step S7, the processing returns to step S3, and the second ultrasound image U2 is reacquired. In this case, the user adjusts the position or the posture of the ultrasound probe 1 in order to appropriately acquire the second ultrasound image U2. Thereafter, the processing of steps S2 to S7 are performed. In this way, the processing of steps S3 to S7 is repeated until it is determined not to reacquire the second ultrasound image U2 in step S7. In this case, the user continues to adjust the position or the posture of the ultrasound probe 1 in order to appropriately acquire the second ultrasound image U2.

By the way, generally, in order to check whether the second ultrasound image U2 representing the apical five-chamber cross section or the apical three-chamber cross section is appropriately acquired, a series of operations of installing a so-called Doppler gate on the left ventricular outflow tract T in the second ultrasound image U2, acquiring a time-varying waveform of the blood flow velocity in the left ventricular outflow tract T using a so-called pulse Doppler method in the Doppler gate, and checking the time-varying waveform is often repeated, and it may take a large amount of time to obtain an appropriate second ultrasound image U2. The user can easily ascertain whether or not the second ultrasound image U2 is appropriately acquired by checking the consistency notified in step S6, and can accurately display the second ultrasound image U2.

In a case in which it is determined not to reacquire the second ultrasound image U2 in step S7, the processing proceeds to step S8. In step S8, the velocity-time integral value calculation unit 30 calculates the velocity-time integral value of the blood flow in the Doppler gate set in the left ventricular outflow tract T shown in the second ultrasound image U2, and the cardiac output calculation unit 31 calculates the cardiac output by using the velocity-time integral value. Since the user can appropriately acquire the second ultrasound image U2 by checking the consistency calculated in step S6, the user can accurately calculate the cardiac output by using the appropriately acquired second ultrasound image U2.

In a case in which the processing of step S8 is completed in this manner, the operation of the ultrasound diagnostic apparatus according to the flowchart of FIG. 9 is completed.

As described above, according to the ultrasound diagnostic apparatus of the embodiment of the present invention, since the consistency calculation unit 28 calculates the consistency between the first ultrasound image U1 and the second ultrasound image U2 by comparing the first measurement value acquired by the first measurement unit 26 with the second measurement value acquired by the second measurement unit 27, and the notification unit 29 notifies the user of the consistency calculated by the consistency calculation unit 28, the first ultrasound image U1 and the second ultrasound image U2 that are consistent with each other can be accurately displayed.

It should be noted that a case has been described in which the transmission and reception circuit 12 is provided in the ultrasound probe 1, but the transmission and reception circuit 12 may be provided in the apparatus main body 2.

In addition, although the image generation unit 21 has been described as being provided in the apparatus main body 2, the image generation unit 21 may be provided in the ultrasound probe 1.

The apparatus main body 2 may be a so-called stationary type, a portable type that is easy to carry, or a so-called handheld type that is configured by, for example, a smartphone or a tablet type computer. As described above, the type of equipment constituting the apparatus main body 2 is not particularly limited.

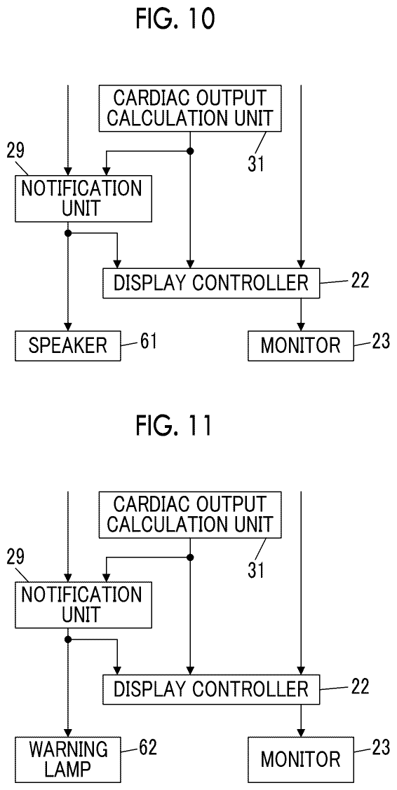

Although the example has been described in which the notification unit 29 notifies the user by displaying on the monitor 23, the method of the notification by the notification unit 29 is not particularly limited thereto. For example, as shown in FIG. 10, in a case in which the ultrasound diagnostic apparatus includes a speaker 61, the notification unit 29 can also output the consistency calculated by the consistency calculation unit 28 from the speaker 61 in a voice.

In addition, for example, as shown in FIG. 11, the ultrasound diagnostic apparatus can also include a warning lamp 62. The notification unit 29 can cause the warning lamp 62 to emit light in a case in which the consistency calculated by the consistency calculation unit 28 is lower than a predetermined consistency threshold value. As shown in FIG. 11, the warning lamp 62 can be provided in the apparatus main body 2, can also be provided in the ultrasound probe 1, and can also be installed independently of the ultrasound probe 1 and the apparatus main body 2.

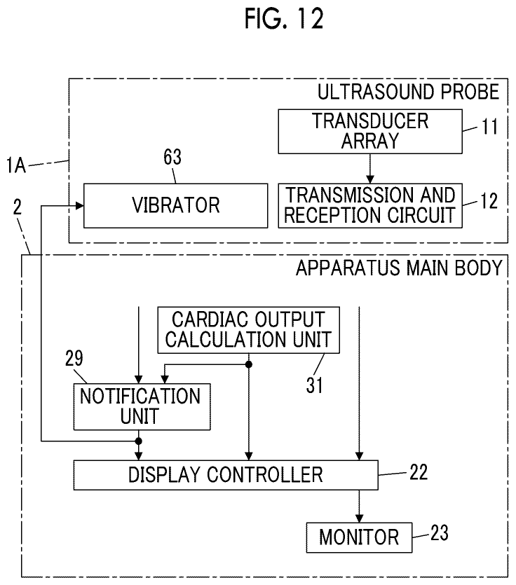

In addition, for example, as shown in FIG. 12, the ultrasound diagnostic apparatus can include an ultrasound probe 1A that has a built-in vibrator 63. As described above, in a case in which the ultrasound probe 1A includes the vibrator 63, the notification unit 29 can cause the vibrator 63 to vibrate in a case in which the consistency calculated by the consistency calculation unit 28 is lower than the predetermined consistency threshold value. The vibrator 63 can be configured by a so-called vibration motor or the like.

Although a case has been described in which the image acquisition unit 34 determines which of the first ultrasound image U1 or the second ultrasound image U2 is acquired based on a recognition result of the image recognition unit 25, the user can also input information on which of the first ultrasound image U1 or the second ultrasound image U2 is acquired via the input device 33. In this case, the first measurement unit 26 and the second measurement unit 27 can determine whether or not to perform the processing with reference to the information input by the user.

By the way, in a case in which so-called papillary muscles are shown in the second ultrasound image U2 representing the apical five-chamber cross section, it can be determined that the second ultrasound image U2 is not appropriately depicted. Therefore, the image recognition unit 25 performs processing of recognizing the papillary muscles from the second ultrasound image U2 by a so-called template matching method or a method of using a trained model in machine learning, and the notification unit 29 can notify the user that the second ultrasound image U2 is not appropriately depicted in a case in which the papillary muscles are recognized by the image recognition unit 25.

In addition, although a case has been described in which the cardiac output is measured in step S8, the processing can be stopped after the measurement of the stroke volume depending on the purpose of the examination or the like, and the processing can also be stopped after the calculation of the velocity-time integral value of the blood flow.

### EXPLANATION OF REFERENCES

1: ultrasound probe
2: apparatus main body
11: transducer array
12: transmission and reception circuit
21: image generation unit
22: display controller
23: monitor
24: image memory
25: image recognition unit
26: first measurement unit
27: second measurement unit
28: consistency calculation unit
29: notification unit
30: velocity-time integral value calculation unit
31: cardiac output calculation unit
32: apparatus controller
33: input device
34: image acquisition unit
35: processor
41: pulser
42: amplifying unit
43: AD conversion unit
44: beam former
45: signal processing unit
46: DSC
47: image processing unit
51: gate setting unit
52: quadrature detection unit
53: high-pass filter
54: fast Fourier transformer
55: Doppler waveform acquisition unit
56: integral value calculation unit
61: speaker
62: warning lamp
63: vibrator
M: message
ML1, ML2: measurement line
T: left ventricular outflow tract
U1: first ultrasound image
U2: second ultrasound image

What is claimed is:

1. An ultrasound diagnostic apparatus comprising:
a processor is configured to:
acquire a first ultrasound image and a second ultrasound image in which first and second cross sections of a heart of a subject that are different from each other are imaged, respectively;
acquire a first measurement value of a predetermined measurement target depicted in the first cross section from the first ultrasound image;
acquire a second measurement value of the predetermined measurement target depicted in the second cross section from the second ultrasound image;
calculate consistency between the first ultrasound image and the second ultrasound image by comparing the first measurement value and the second measurement value; and
notify a user of the consistency.

2. The ultrasound diagnostic apparatus according to claim 1,
wherein the processor is configured to:
acquire the first ultrasound image in which a parasternal left ventricular long-axis cross section is set as the first cross section and the second ultrasound image in which an apical five-chamber cross section or an apical three-chamber cross section is set as the second cross section;
measure a left ventricular outflow tract diameter on the first ultrasound image as the first measurement value; and
measure a left ventricular outflow tract diameter on the second ultrasound image as the second measurement value.

3. The ultrasound diagnostic apparatus according to claim 1,
wherein the processor is configured to calculate a difference or a ratio between the first measurement value and the second measurement value as the consistency.

4. The ultrasound diagnostic apparatus according to claim 2,
wherein the processor is configured to calculate a difference or a ratio between the first measurement value and the second measurement value as the consistency.

5. The ultrasound diagnostic apparatus according to claim 1, further comprising:
a monitor,
wherein the processor is configured to display the consistency on the monitor.

6. The ultrasound diagnostic apparatus according to claim 2, further comprising:
a monitor,
wherein the processor is configured to display the consistency on the monitor.

7. The ultrasound diagnostic apparatus according to claim 3, further comprising:
a monitor,
wherein the processor is configured to display the consistency on the monitor.

8. The ultrasound diagnostic apparatus according to claim 1, further comprising:
a speaker,
wherein the processor is configured to output the consistency as sound from the speaker.

9. The ultrasound diagnostic apparatus according to claim 2, further comprising:
a speaker,
wherein the processor is configured to output the consistency as sound from the speaker.

10. The ultrasound diagnostic apparatus according to claim 3, further comprising:
a speaker,
wherein the processor is configured to output the consistency as sound from the speaker.

11. The ultrasound diagnostic apparatus according to claim 1, further comprising:
a lamp,
wherein the processor is configured to cause the lamp to emit light while the consistency is being lower than a predetermined threshold value.

12. The ultrasound diagnostic apparatus according to claim 2, further comprising:
a lamp,
wherein the processor is configured to cause the lamp to emit light while the consistency is being lower than a predetermined threshold value.

13. The ultrasound diagnostic apparatus according to claim 3, further comprising:
a lamp,
wherein the processor is configured to cause the lamp to emit light while the consistency is being lower than a predetermined threshold value.

14. The ultrasound diagnostic apparatus according to claim 1, further comprising:
an ultrasound probe,
wherein the ultrasound probe has a built-in vibrator, and
the processor is configured to cause the vibrator to vibrate while the consistency is being lower than a predetermined threshold value.

15. The ultrasound diagnostic apparatus according to claim 2, further comprising:
an ultrasound probe,
wherein the ultrasound probe has a built-in vibrator, and
the processor is configured to cause the vibrator to vibrate while the consistency is being lower than a predetermined threshold value.

16. The ultrasound diagnostic apparatus according to claim 3, further comprising:
an ultrasound probe,
wherein the ultrasound probe has a built-in vibrator, and
the processor is configured to cause the vibrator to vibrate while the consistency is being lower than a predetermined threshold value.

17. The ultrasound diagnostic apparatus according to claim 1,
wherein the processor is configured to:
calculate a velocity-time integral value of blood flow in a left ventricular outflow tract from the second ultrasound image;
calculate a cardiac output based on the left ventricular outflow tract diameter as the first measurement value and the velocity-time integral value; and
notify the user upon determining that the cardiac output deviates from a predetermined reference value.

18. The ultrasound diagnostic apparatus according to claim 2,
wherein the processor is configured to:
calculate a velocity-time integral value of blood flow in a left ventricular outflow tract from the second ultrasound image;
calculate a cardiac output based on the left ventricular outflow tract diameter as the first measurement value and the velocity-time integral value; and
notify the user upon determining that the cardiac output deviates from a predetermined reference value.

19. The ultrasound diagnostic apparatus according to claim 3,
wherein the processor is configured to:
calculate a velocity-time integral value of blood flow in a left ventricular outflow tract from the second ultrasound image;
calculate a cardiac output based on the left ventricular outflow tract diameter as the first measurement value and the velocity-time integral value; and
notify the user upon determining that the cardiac output deviates from a predetermined reference value.

20. A control method of an ultrasound diagnostic apparatus, the method comprising:
acquiring a first ultrasound image and a second ultrasound image in which first and second cross sections of a heart of a subject that are different from each other are imaged, respectively;
acquiring a first measurement value of a predetermined measurement target depicted in the first cross section from the first ultrasound image;
acquiring a second measurement value of the predetermined measurement target depicted in the second cross section from the second ultrasound image;
calculating consistency between the first ultrasound image and the second ultrasound image by comparing the first measurement value and the second measurement value that are acquired; and
notifying a user of the calculated consistency.
